(19) United States
(12) Patent Application Publication
(10) Pub. No.: US 2025/0352770 A1
(43) Pub. Date: Nov. 20, 2025

(54) Guidewire Surface Modification to Reduce Drag Force

(71) Applicant: Bard Access Systems, Inc., Salt Lake City, UT (US)

(72) Inventors: Chase Provost, West Bountiful, UT (US); Amanda M. Lingle, Salt Lake City, UT (US); Sonia Maria Osuna, Salt Lake City, UT (US); Marcus Jones, Layton, UT (US)

(21) Appl. No.: 19/208,478

(22) Filed: May 14, 2025

Related U.S. Application Data
(60) Provisional application No. 63/649,782, filed on May 20, 2024.

(51) Int. Cl. A61M 25/09

(52) U.S. Cl. CPC A61M 25/09; A61M 2025/09058; A61M 2025/09108

(57) ABSTRACT

A guidewire having a modified surface to reduce drag force between the outer surface of the guidewire and an inner surface of the catheter lumen. Preloaded catheter systems include a guidewire disposed within a lumen of the catheter. The guidewire is equal to, or slightly smaller than an inner diameter of the catheter lumen to impart columnar strength to the catheter during placement. However, due to increase contact surface area between the guidewire and the catheter lumen, the guidewire can stick within catheter lumen requiring increase force to slide the guidewire relative to the catheter. Surface modifications on the guidewire can reduce the contact surface are between the guidewire and catheter resulting in a reduced drag force therebetween. A plurality of longitudinal grooves, disposed in a regular or irregular pattern, and reduce drag force without affecting the flexible properties of the guidewire.

### PRIORITY

This application claims the benefit of priority to U.S. Provisional Application No. 63/649,782, filed May 20, 2024, which is incorporated by reference in its entirety into this application.

### BACKGROUND

Placing catheters within a patient often includes the use of a guidewire. The guidewire is advanced to a target location, e.g., within a vasculature, and the catheter is advanced over the guidewire to the target location, often with the guidewire passing through a lumen of the catheter. The guidewire provides columnar strength to the catheter during placement to prevent collapse or buckling of the catheter. Often the outer diameter of the guidewire is the same, or only slightly smaller, than the inner diameter of the catheter lumen, or catheter tip, to impart the increased rigidity of the guidewire to the catheter.

Previous catheter systems have typically involved using guidewires with smooth outer surfaces to facilitate insertion and navigation through the catheter lumen. These smooth guidewires have limitations when it comes to reducing drag force within the catheter lumen. The smooth outer surface matches the smooth inner surface of the catheter lumen providing a large contact surface area therebetween, which increases drag force. For preloaded catheters, where the guidewire is disposed within the catheter lumen during manufacture, the guidewire can reside there for a prolonged period of time during transport and storage. As such, during use, the guidewire can “stick” to the catheter and requires the user to break the contact between the guidewire and the catheter to ensure a smooth movement. Even if the user can work loose the guidewire prior to use, which may not always be possible, the smooth guidewire can still display increased drag force on the catheter.

In some instances, attempts have been made to reduce friction within catheter systems by incorporating lubricious coatings on the guidewire surfaces. While these coatings can help in reducing friction to some extent, they may wear off or dry out over time, leading to the reemergence of drag forces within the catheter lumen. Additionally, the application of coatings may introduce complexities in the manufacturing process and may not provide a permanent solution to the drag force issue experienced during catheterization procedures.

Disclosed herein are guidewire systems and associated methods directed to address the foregoing.

### SUMMARY

In some aspects, the techniques described herein relate to a catheter system including, a catheter defining a catheter lumen, and a guidewire disposed in the catheter lumen, an outer diameter of the guidewire equal to or less than an inner diameter of the catheter lumen, an outer surface of the guidewire including a surface modification including a plurality of grooves configured to provide a relatively lower drag force on an inner surface of the catheter lumen relative to a guidewire having a relatively smooth outer surface.

In some aspects, the techniques described herein relate to a catheter system, wherein the plurality of grooves extend parallel to a central longitudinal axis of the guidewire.

In some aspects, the techniques described herein relate to a catheter system, wherein the plurality of grooves are disposed annularly evenly about a central longitudinal axis and extend parallel to each other.

In some aspects, the techniques described herein relate to a catheter system, wherein the plurality of grooves extend helically about a central longitudinal axis.

In some aspects, the techniques described herein relate to a catheter system, wherein a first arc distance between a trough of a first groove of the plurality of grooves and a trough of a second groove of the plurality of grooves disposed adjacent thereto, is equal to a second arc distance between the trough of the second groove and a trough of a third groove of the plurality of grooves disposed adjacent to the second groove.

In some aspects, the techniques described herein relate to a catheter system, wherein a first arc distance between a trough of a first groove of the plurality of grooves and a trough of a second groove of the plurality of grooves disposed adjacent thereto, is different from a second arc distance between the trough of the second groove and a trough of a third groove of the plurality of grooves disposed adjacent to the second groove.

In some aspects, the techniques described herein relate to a catheter system, wherein the plurality of grooves are disposed annularly irregularly about a central longitudinal axis.

In some aspects, the techniques described herein relate to a catheter system, wherein a groove depth is between 15 μm and 50 μm.

In some aspects, the techniques described herein relate to a catheter system, wherein the plurality of grooves are formed by one of scoring, laser marking, directional mill finish, extrusion, abrasive paper, sandpaper, bead blasting, grit blasting, wire brushing, chemical etching.

In some aspects, the techniques described herein relate to a catheter system, wherein the plurality of grooves are formed using abrasive paper having a grit size of between 100 and 600.

In some aspects, the techniques described herein relate to a catheter system, wherein the plurality of grooves are formed using abrasive paper having a grit size of 320.

In some aspects, the techniques described herein relate to a method of manufacturing a catheter system including, forming a catheter defining a catheter lumen having an inner diameter, forming a guidewire extending longitudinally between a distal end and a proximal end and having an outer dimeter equal to or less than the inner diameter of the catheter lumen, forming a plurality of grooves in an outer surface of the guidewire, the plurality of grooves configured to provide a relatively lower drag force on an inner surface of the catheter lumen relative to a guidewire having a relatively smooth outer surface, and placing the guidewire in the catheter lumen.

In some aspects, the techniques described herein relate to a method, wherein forming the plurality of grooves includes forming the plurality of grooves to extend parallel to a central longitudinal axis of the guidewire.

In some aspects, the techniques described herein relate to a method, wherein forming the plurality of grooves includes forming the plurality of grooves to be disposed annularly evenly about a central longitudinal axis and extend parallel to each other.

In some aspects, the techniques described herein relate to a method, wherein forming the plurality of grooves includes forming the plurality of grooves to extend helically about a central longitudinal axis.

In some aspects, the techniques described herein relate to a method, wherein a first arc distance between a trough of a first groove of the plurality of grooves and a trough of a second groove of the plurality of grooves disposed adjacent thereto, is different from a second arc distance between the trough of the second groove and a trough of a third groove of the plurality of grooves disposed adjacent to the second groove.

In some aspects, the techniques described herein relate to a method, wherein forming the plurality of grooves includes forming the plurality of grooves irregularly about a circumference of the guidewire.

In some aspects, the techniques described herein relate to a method, wherein forming the plurality of grooves includes forming a first groove of the plurality of grooves to extend at an angle relative to a second groove of the plurality of grooves.

In some aspects, the techniques described herein relate to a method, wherein forming the plurality of grooves includes forming the plurality of grooves with a groove depth of between 15 μm and 50 μm.

In some aspects, the techniques described herein relate to a method, wherein the plurality of grooves are formed by one of scoring, laser marking, directional mill finish, extrusion, abrasive paper, sandpaper, bead blasting, grit blasting, wire brushing, chemical etching.

In some aspects, the techniques described herein relate to a method, wherein the plurality of grooves are formed using abrasive paper having a grit size of between 100 and 600.

In some aspects, the techniques described herein relate to a method, wherein the plurality of grooves are formed using abrasive paper having a grit size of 320.

In some aspects, the techniques described herein relate to a guidewire including, a body extending along a longitudinal axis between a distal end and a proximal end, and one or more grooves formed on an outer surface of the body.

In some aspects, the techniques described herein relate to a guidewire, wherein the one or more grooves includes a groove depth of between 15 μm and 50 μm.

In some aspects, the techniques described herein relate to a guidewire, wherein the one or more grooves extend parallel to a central longitudinal axis of the guidewire.

In some aspects, the techniques described herein relate to a guidewire, wherein the one or more grooves are disposed evenly about a circumference of the body and extend parallel to each other.

In some aspects, the techniques described herein relate to a guidewire, wherein the one or more grooves extend helically about a central longitudinal axis.

In some aspects, the techniques described herein relate to a guidewire, wherein the one or more grooves are disposed irregularly about a circumference of the body of the guidewire.

In some aspects, the techniques described herein relate to a guidewire, wherein the one or more grooves are formed by one of scoring, laser marking, directional mill finish, extrusion, abrasive paper, sandpaper, bead blasting, grit blasting, wire brushing, chemical etching.

In some aspects, the techniques described herein relate to a guidewire, wherein the one or more grooves are formed using abrasive paper having a grit size of between 100 and 600.

In some aspects, the techniques described herein relate to a guidewire, wherein the one or more grooves are formed using abrasive paper having a grit size of 320.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a catheter system including a catheter and a guidewire, in accordance with embodiments disclosed herein.

FIG. 2A shows a cross-section view of a guidewire disposed in a lumen of a catheter, in accordance with embodiments disclosed herein.

FIG. 2B shows a cross-section view of a guidewire including a coating disposed in a lumen of a catheter, in accordance with embodiments disclosed herein.

FIG. 3A shows a cross-section view of a guidewire including a plurality of grooves disposed evenly about a central longitudinal axis, in accordance with embodiments disclosed herein.

FIG. 3B shows a cross-section view of a guidewire including one or more grooves disposed evenly about a central longitudinal axis, in accordance with embodiments disclosed herein.

FIG. 3C shows close up detail of a surface of the guidewire of FIG. 3A, in accordance with embodiments disclosed herein.

FIG. 3D shows a cross-section view of a guidewire including a plurality of grooves disposed irregularly about a central longitudinal axis, in accordance with embodiments disclosed herein.

FIG. 4A shows a side view of a guidewire including a plurality of grooves extending parallel to a central longitudinal axis, in accordance with embodiments disclosed herein.

FIG. 4B shows a side view of a guidewire including a plurality of grooves extending helically about a central longitudinal axis, in accordance with embodiments disclosed herein.

FIG. 4C shows a side view of a guidewire including a plurality of grooves extending longitudinally and extending at an angle relative to each other, in accordance with embodiments disclosed herein.

FIG. 5 shows a chart comparing the maximum force in pound-force (lbf.) required to move a guidewire relative to a catheter when disposed within a lumen of the catheter for guidewires having different surface modifications, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “right,” “left,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter or system disclosed herein includes a portion of the catheter or system intended to be near or relatively nearer to a clinician when the catheter or system is used on a patient. Likewise, a “proximal length” of, for example, the catheter or system includes a length of the catheter or system intended to be near or relatively nearer to the clinician when the catheter or system is used on the patient. A “proximal end” of, for example, the catheter or system includes an end of the catheter or system intended to be near or relatively nearer to the clinician when the catheter or system is used on the patient. The proximal portion, the proximal end portion, or the proximal length of the catheter or system can include the proximal end of the catheter or system; however, the proximal portion, the proximal end portion, or the proximal length of the catheter or system need not include the proximal end of the catheter or system. That is, unless context suggests otherwise, the proximal portion, the proximal end portion, or the proximal length of the catheter or system is not necessarily a terminal portion or terminal length of the catheter or system.

With respect to “distal,” a “distal portion” or a “distal end portion” of, for example, a catheter or system disclosed herein includes a portion of the catheter or system intended to be near or relatively nearer to a patient when the catheter or system is used on a patient. Likewise, a “distal length” of, for example, the catheter or system includes a length of the catheter or system intended to be near or relatively nearer to the patient when the catheter or system is used on the patient. A “distal end” of, for example, the catheter or system includes an end of the catheter or system intended to be near or relatively nearer to the patient when the catheter or system is used on the patient. The distal portion, the distal end portion, or the distal length of the catheter or system can include the distal end of the catheter or system; however, the distal portion, the distal end portion, or the distal length of the catheter or system need not include the distal end of the catheter or system. That is, unless context suggests otherwise, the distal portion, the distal end portion, or the distal length of the catheter or system is not necessarily a terminal portion or terminal length of the catheter or system.

Unless defined otherwise, all technical and scientific terms used herein have the same meaning as commonly understood by those of ordinary skill in the art.

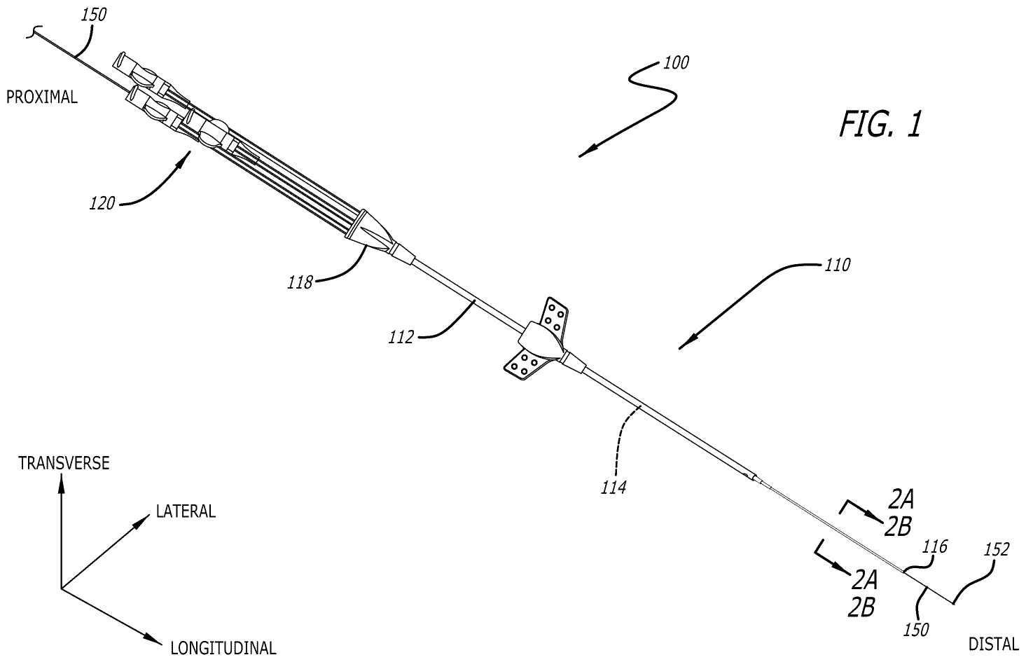

FIG. 1 shows an exemplary catheter and guidewire system (“system”) 100 generally including a catheter 110 and guidewire 150. The catheter 110 generally includes a catheter tube 112 extending to a distal tip 116 and defining one or more catheter lumen 114 communicating with an opening disposed distally. A proximal end of the catheter tube 112 is supported by a catheter hub 118. The catheter hub 118 can include one or more extension legs 120 extending proximally. Each extension leg of the one or more extension legs 120 can provide fluid communication with a lumen 114 of the catheter tube 112. The guidewire 150 can be disposed within a lumen 114 of the catheter and can extend from a proximal end to a distal tip 116 of the catheter 110.

As will be appreciated, the catheter 110 is exemplary and not intended to be limiting. Exemplary catheters 110 can also include central venous catheters (CVC), peripherally inserted central catheters (PICC), rapidly insertable central catheters (RICC), peripheral intravenous catheters (PIV), intravenous (IV) catheters, midline catheters, or the like. The catheter 110 can be formed of a plastic, polymer, elastomer, rubber, silicone rubber, or similar suitable material configured to be flexible enough to negotiate tortuous vascular pathways.

The guidewire 150 can be formed of a plastic, polymer, metal, alloy, composite, super-elastic material, shape memory material, Nitinol, or the like. The guidewire 150 provides increased columnar strength to mitigate kinking or collapsing when an axial force is applied thereto and urged through tortuous vascular pathways to a target location. Once a distal tip 152 of the guidewire 150 is disposed at the target location, the catheter 110 can be advanced over the guidewire 150 so that the catheter distal tip 116 is disposed at the target location.

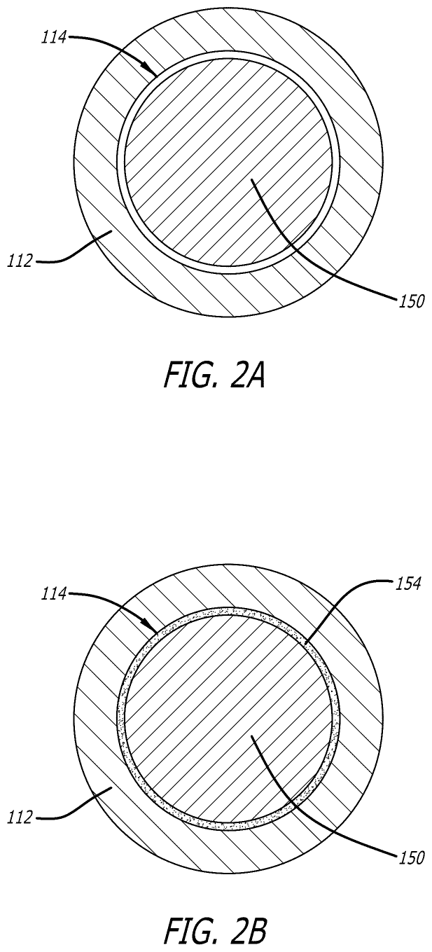

As shown in FIG. 2A, an outer diameter of the guidewire 150 can be equal to, or slightly smaller than the inner diameter of the catheter lumen 114. As such, an outer surface of the guidewire 150 contacts an inner surface of the catheter lumen 114 and provides support to the catheter 110. The guidewire 150 then imparts the relatively increased rigidity, and/or columnar strength of the guidewire 150 on to the catheter 110 to mitigate collapse or kinking of the catheter 110 during placement. Bending, collapse, or kinking of the catheter 110 can damage the catheter 110 reducing the efficacy of the device, shortening the device lifespan, and/or leading to failure of the device.

As shown in FIG. 1, the catheter 110 can be provided as “preloaded” with a guidewire 150 already disposed within the catheter lumen 114. Advantageously, these preloaded catheters save time during placement by removing the step of loading the guidewire into the catheter lumen. Further, these preloaded catheters mitigate any damage that might occur during a loading process carried out by a technician. As such, the guidewire 150 can be advanced through catheter lumen 114 to a target location and the catheter 110 can then be advanced over the guidewire 150 to the target location.

As shown in FIG. 2A, a guidewire 150 defining a relatively smoother outer surface provides a relatively large surface area that contacts the inner surface of the catheter lumen 114. As such, the large contact area between the guidewire 150 and the catheter 110 provides increased friction therebetween. The increase friction or drag force can affect the movement of the guidewire 150 relative to the catheter 110 during use, which can affect the speed and efficiency of the placement process. For example, the guidewire 150 can stick to the inner wall of the catheter lumen 114, requiring the technician to apply increased force between the catheter 110 and guidewire 150 to overcome the drag force. When the contact between the guidewire and catheter is overcome, the increase force can result in the catheter and/or guidewire pulling away from each other suddenly resulting in accidental misplacement of the guidewire and/or catheter. Alternatively, the drag force can result in a less consistent movement between the guidewire and catheter.

In an embodiment, as shown in FIG. 2B, the guidewire 150 can further include one or more coatings 154 disposed on at least a portion of an outer surface thereof. In an embodiment, the one or more coatings 154 can be disposed over an entire surface of the guidewire 150. The coating 154 can provide increased contact area between the guidewire 150 and the catheter 110, providing increased friction therebetween. In an embodiment, the coating 154 can provide increased surface tension between the guidewire 150 and the catheter 110 leading to increased friction and/or adhesion. In an embodiment, between manufacture and use, i.e., over the course of storage and transport, the coating 154 may dry out a little causing increased friction and/or adhesion between the guidewire 150 and catheter 110. In an embodiment, between manufacture and use, the chemicals of the coating 154 can react with the material of the catheter 110 causing increased friction and/or adhesion between the guidewire 150 and catheter 110.

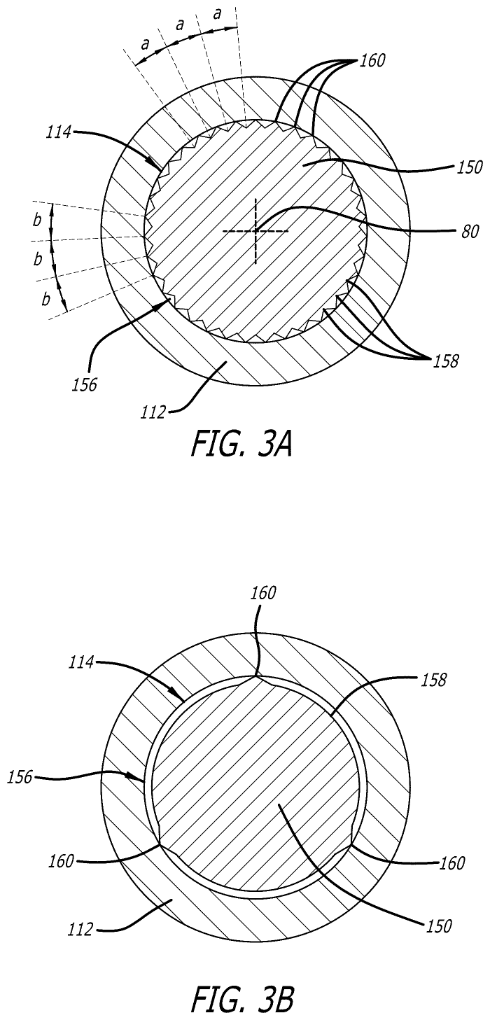

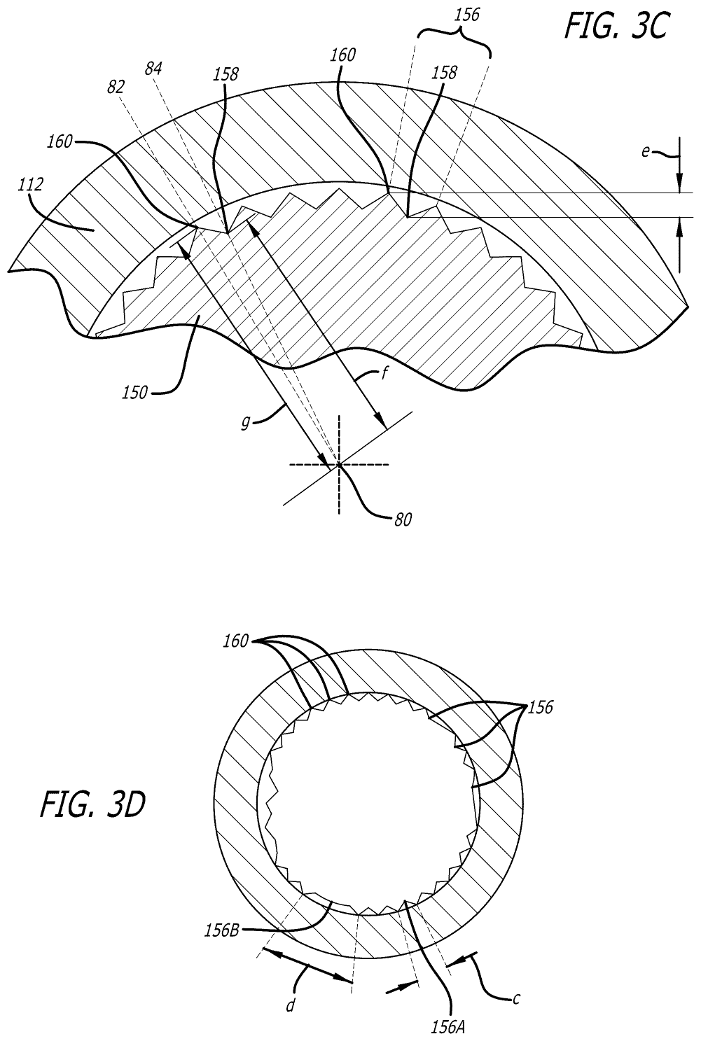

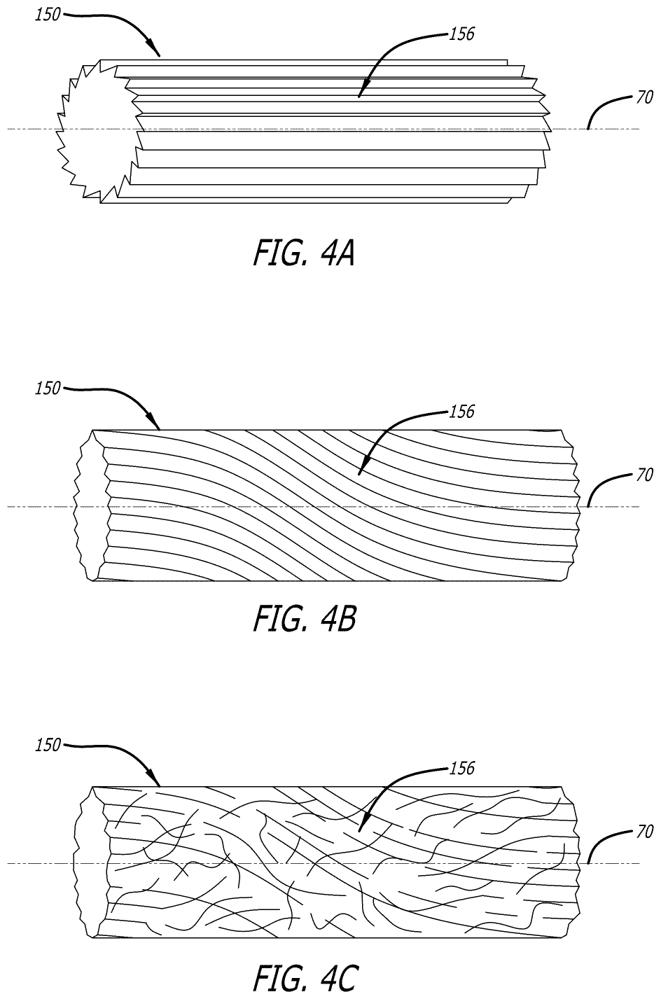

FIGS. 3A-4C show embodiments of a guidewire 150 including one or more surface modifications configured to reduce a contact surface area between the outer surface of the guidewire 150 and the inner surface of the catheter lumen 114 to reduce a drag force therebetween. In an embodiment, the surface modifications of the guidewire 150 includes one or more grooves, striations, and/or a roughened outer surface. As such, the modified surface provides a reduced surface area in contact with the inner surface of the catheter lumen 114 that provides a reduced drag force between the guidewire 150 and the catheter 110.

In an embodiment, as shown in FIGS. 3A and 4A, the outer surface of the guidewire 150 includes one or more grooves 156 extending longitudinally. In an embodiment, the one or more grooves 156 can extend over a portion of the longitudinal length of the guidewire 150. In an embodiment, the one or more grooves 156 can extend over an entire longitudinal length of the guidewire 150. In an embodiment, as shown in FIG. 3A, the one or more grooves 156 can be disposed annularly along a circumference of the guidewire 150. In an embodiment, the one or more grooves 156 can be disposed evenly about the circumference of the guidewire 150.

In an embodiment, as shown in FIGS. 3A and 3C, adjacent grooves 156 can form a peak 160 therebetween. The peak 160 being a radially outer-most surface of the guidewire 150 between the adjacent grooves 156. As will be appreciated, where the one or more grooves 156 are disposed evenly about the perimeter of the guidewire 150, the associated peaks 160 disposed between the grooves 156 are also disposed evenly about the perimeter. Further, a radially inner-most surface of the groove 156 is a trough 158. In an embodiment, the trough 158 of each groove of the one or more grooves 156 can be disposed evenly about the perimeter of the guidewire 150.

As shown in FIG. 3A, in an embodiment, a trough 158 of a first groove 156 can be disposed equidistant, e.g., by a distance (a), from a trough 158 of an adjacent groove 156 in a first direction and from a trough 158 of an adjacent groove 156 in a second direction about the perimeter of the guidewire 150. Further, a peak 160 of a first groove 156 can be disposed equidistant, e.g., by a distance (b), from a peak 160 of an adjacent groove 156 in a first direction and from a peak 160 of an adjacent groove 156 in a second direction about the perimeter of the guidewire 150. The distance (a) between the troughs 158 can be equal to the distance (b) between the peaks 160. Advantageously, the peaks 160 are the radially outer-most surface of the guidewire 150 and provide a reduced contact surface area between the guidewire 150 and the inner surface of the catheter lumen 114. In an embodiment, an inflection point at the peak 160 can be angled to provide a reduced contact surface area between the guidewire 150 and the inner wall of the catheter lumen 114. In an embodiment, the inflection point at the peak can be rounded to reduce wear and damage to the inner wall of the catheter lumen 114.

FIG. 3C shows close detail of a portion of the guidewire 150 and the catheter 110 of FIG. 3A. As shown, a depth (e) of a groove 156 is a radial distance between a peak 160 and an adjacent trough 158. Advantageously, the plurality of grooves 156 allow the depth of the grooves 156 to be relatively shallow, while the peaks 160 contact inner surface of the catheter lumen 114, maintaining the guidewire 150 in a radially central position within the catheter lumen 114, i.e., disposed centrally along the lateral and transverse axes of the catheter lumen 114. Further, the increased number of peaks 160 provide a more dispersed pressure on the inner wall of the catheter lumen 114 providing reduced wear. In an embodiment, the depth (e) of the grooves 156 range from between 1 μm and 80 μm. In an embodiment, the depth (e) of the grooves 156 range from between 15 μm and 50 μm. However, greater or lesser groove depths are also contemplated.

To note, the groove depth (e) is important since it is configured to mitigate differences in flexibility between the peaks 160 and the troughs 158. For example, with continued reference to FIG. 3C, a first axis of flexion 82 aligned with a peak 160 extends from an axial center point 80 of the guidewire 150 through the peak 160. A second axis of flexion 84 aligned with a trough 158 extends from an axial center point 80 of the guidewire 150 through the trough 158. The first axis of flexion 82 provides relatively more rigid, or less flexibility, relative to the second axis of flexion 84 since there is more material, distance (g), aligned with the first axis of flexion 84 than with the second axis of flexion (f). Accordingly, where the depth (e) of the groove 156, i.e., the difference between distance (g) and distance (f), is significantly increased, the greater the mechanical differences between the first axis of flexion 82 and the second axis of flexion 84 which can affect the performance of the guidewire 150. Accordingly, the groove depth (e) is configured to reduce a surface contact area while still providing uniform mechanical properties to the guidewire 150.

In an embodiment, as shown in FIG. 3B, the guidewire can include as few as three grooves 156, defining only three peaks 160 disposed about the perimeter of the guidewire 150. Advantageously, the reduced number of peaks 160 also reduces the contact surface area yet further, providing even less drag force between the guidewire 150 and the catheter.

In an embodiment, as shown in FIG. 3D, the one or more grooves 156 can be disposed irregularly about the perimeter of the guidewire 150. For example, the guidewire 150 can include one or more grooves 156 having different arc distances between a trough 158 of a first groove 156 and a trough 158 of an adjacent groove 156. Similarly, the guidewire 150 can include one or more grooves 156 having different arc distances between a peak 160 of a first groove 156 and a peak 160 of an adjacent groove 156.

In an embodiment, as shown in FIG. 3D, an arc radius between adjacent peaks 160 can vary providing grooves 156 with different arc distance. For example, a first groove 156A can define a first arc distance (c), i.e., having adjacent peaks 160 disposed at a first arc distance (c) apart. Further, a second groove 156B can define a second arc distance (b), i.e., having adjacent peaks 160 disposed at a second arc distance (d) apart. The first arc distance (a) being different from the second arc distance (d). Advantageously, the irregular positioning of one or more of the grooves 156 location on the perimeter, the arc distance between adjacent troughs 158, the arc distance between adjacent peaks 160, the groove depth (e), or combinations thereof, can provide a reduced contact surface area between the guidewire 150 and the inner surface of the catheter lumen 114 while maintaining uniform mechanical properties of the guidewire 150.

In an embodiment, as shown in FIG. 4A, the one or more grooves 156 extend parallel to each other and to the longitudinal axis 70 of the guidewire 150. In an embodiment, as shown in FIG. 4B, the one or more grooves 156 extend parallel to each other but extend at an angle to the longitudinal axis 70 of the guidewire 150 to extend helically about the guidewire 150. The angle of the helical grooves 156 can extend between 1° and 89° degrees relative to the longitudinal axis 70. The angle of the helical grooves 156 can extend between 1° and 45° degrees relative to the longitudinal axis 70. However, greater or lesser angles are also contemplated. Advantageously, the helical configuration of the grooves prevents placing too much wear on a specific area of the inner surface of the catheter lumen 114 and instead spreads the wear evenly over the inner surface while reduce overall contact surface area.

In an embodiment, as shown in FIG. 4C, the one or more grooves 156 extend at an angle to the longitudinal axis 70 of the guidewire 150. In an embodiment, the one or more grooves 156 extend at an angle relative to each other. In an embodiment, an axis of a first groove 156 can intersect an axis of a second groove 156. As such, the grooves 156 can be provided as a randomized disbursement of grooves 156 over the surface of the guidewire 150.

In an embodiment, the one or more grooves 156 are formed on the surface of the guidewire 150 using one or more of scoring, laser marking, directional mill finish, extrusion, abrasive paper, sandpaper, bead blasting, grit blasting, wire brushing, chemical etching, combinations thereof, or the like.

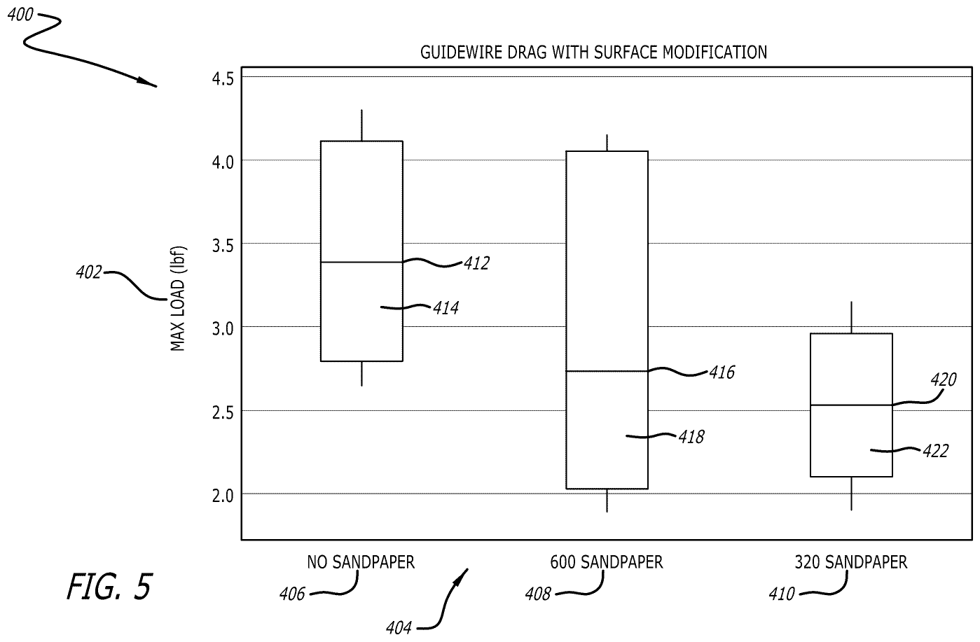

FIG. 5 shows evidence of unexpected results when comparing the guidewire drag force for different guidewires having differing surface modifications. Exemplary guidewires include a control guidewire having no surface modifications, a first guidewire having a first surface modification and a second guidewire having a second surface modification. FIG. 5 shows a graph 400 of guidewire drag force with surface modifications. The y-axis 402 shows the maximum amount of load, in pound-force (lbf.), required before the guidewire 150 of a preloaded catheter system 100 is moved relative to the catheter 110. The x-axis 404 details the result for the three different guidewires. A control guidewire 406, termed “No Sandpaper,” has no surface modifications and defines a smooth outer surface, as shown in FIG. 2A. A first test guidewire 408, termed “600 Sandpaper,” has a first surface modification and includes one or more grooves 156 formed on the surface of the guidewire 150 using a relatively high grit sandpaper, e.g., a 600-grit sandpaper. A second test guidewire 410, termed “320 Sandpaper,” has a second surface modification and includes one or more grooves 156 formed on the surface of the guidewire 150 using a relatively low grit sandpaper, e.g., a 320-grit sandpaper, defining a relatively coarser grit than that used to form the “600 Sandpaper” of the first test guidewire 408.

As shown in the chart of FIG. 5, the control guidewire 406 required a median force 412 of about 3.4 lbf. with an interquartile range 414 of between about 2.8-4.1 lbf. of force before the guidewire moved relative to the catheter. The first test guidewire 408 required a median force 416 of about 2.7 lbf. with an interquartile range 418 of between about 2.05-4.05 lbf. of force before the guidewire moved relative to the catheter. The second test guidewire 420 required a median force of about 2.5 lbf. with an interquartile range 422 of between about 2.1-2.9 lbf. of force before the guidewire moved relative to the catheter. As such, the second test guidewire 410 having a relatively coarse surface modification required significantly less force before the guidewire moved relative to the catheter, and as such, provided significantly lower drag force.

Accordingly, the first test guidewire 408 had some surface modifications, the groove depth (e) provided some reduction in surface drag force. The second test guidewire 410 having relatively deeper groove depth (e) compared with the first test guidewire 408 provided greater reduction in surface drag force. The relatively coarse surface modifications of the second test guidewire 410 using the 320-grit sandpaper was sufficient to provide a reduction in contact surface area, resulting in a reduced drag force, without modifying the overall mechanical properties of the guidewire 150.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter system, comprising:
a catheter defining a catheter lumen; and
a guidewire disposed in the catheter lumen, an outer diameter of the guidewire equal to or less than an inner diameter of the catheter lumen, an outer surface of the guidewire including a surface modification including a plurality of grooves configured to provide a relatively lower drag force on an inner surface of the catheter lumen relative to a guidewire having a relatively smooth outer surface.

2. The catheter system according to claim 1, wherein the plurality of grooves extends parallel to a central longitudinal axis of the guidewire.

3. The catheter system according to claim 1, wherein the plurality of grooves is disposed annularly evenly about a central longitudinal axis and extend parallel to each other.

4. The catheter system according to claim 1, wherein the plurality of grooves extends helically about a central longitudinal axis.

5. The catheter system according to claim 1, wherein a first arc distance between a trough of a first groove of the plurality of grooves and a trough of a second groove of the plurality of grooves disposed adjacent thereto, is equal to a second arc distance between the trough of the second groove and a trough of a third groove of the plurality of grooves disposed adjacent to the second groove.

6. The catheter system according to claim 1, wherein a first arc distance between a trough of a first groove of the plurality of grooves and a trough of a second groove of the plurality of grooves disposed adjacent thereto, is different from a second arc distance between the trough of the second groove and a trough of a third groove of the plurality of grooves disposed adjacent to the second groove.

7. The catheter system according to claim 1, wherein the plurality of grooves are disposed annularly irregularly about a central longitudinal axis.

8. The catheter system according to claim 1, wherein a groove depth is between 15 μm and 50 μm.

9. The catheter system according to claim 1, wherein the plurality of grooves is formed by one of scoring, laser marking, directional mill finish, extrusion, abrasive paper, sandpaper, bead blasting, grit blasting, wire brushing, chemical etching.

10. The catheter system according to claim 9, wherein the plurality of grooves are formed using abrasive paper having a grit size of between 100 and 600.

11. The catheter system according to claim 9, wherein the plurality of grooves are formed using abrasive paper having a grit size of 320.

12. A method of manufacturing a catheter system, comprising:
forming a catheter defining a catheter lumen having an inner diameter;
forming a guidewire extending longitudinally between a distal end and a proximal end and having an outer dimeter equal to or less than the inner diameter of the catheter lumen;
forming a plurality of grooves in an outer surface of the guidewire, the plurality of grooves configured to provide a relatively lower drag force on an inner surface of the catheter lumen relative to a guidewire having a relatively smooth outer surface; and
placing the guidewire in the catheter lumen.

13. The method according to claim 12, wherein forming the plurality of grooves includes forming the plurality of grooves to extend parallel to a central longitudinal axis of the guidewire.

14. The method according to claim 12, wherein forming the plurality of grooves includes forming the plurality of grooves to be disposed annularly evenly about a central longitudinal axis and extend parallel to each other.

15. The method according to claim 12, wherein forming the plurality of grooves includes forming the plurality of grooves to extend helically about a central longitudinal axis.

16. The method according to claim 12, wherein a first arc distance between a trough of a first groove of the plurality of grooves and a trough of a second groove of the plurality of grooves disposed adjacent thereto, is different from a second arc distance between the trough of the second groove and a trough of a third groove of the plurality of grooves disposed adjacent to the second groove.

17. The method according to claim 12, wherein forming the plurality of grooves includes forming the plurality of grooves irregularly about a circumference of the guidewire.

18. The method according to claim 12, wherein forming the plurality of grooves includes forming a first groove of the plurality of grooves to extend at an angle relative to a second groove of the plurality of grooves.

19. The method according to claim 12, wherein forming the plurality of grooves includes forming the plurality of grooves with a groove depth of between 15 μm and 50 μm.

20. The method according to claim 12, wherein the plurality of grooves are formed by one of scoring, laser marking, directional mill finish, extrusion, abrasive paper, sandpaper, bead blasting, grit blasting, wire brushing, chemical etching.

21. The method according to claim 20, wherein the plurality of grooves are formed using abrasive paper having a grit size of between 100 and 600.

22. The method according to claim 20, wherein the plurality of grooves are formed using abrasive paper having a grit size of 320.

23-31. (canceled)
